(19) United States
(12) Patent Application Publication
(10) Pub. No.: US 2006/0122631 A1
(43) Pub. Date: Jun. 8, 2006

(54) Skin Cleaner

(76) Inventor: M. Glen Kertz, El Paso, TX (US)

Correspondence Address: CONLEY ROSE, P.C., P. O. BOX 3267, HOUSTON, TX, 77253-3267, US

(21) Appl. No.: 11/343,036

(22) Filed: Jan. 30, 2006

Related U.S. Application Data
(63) Continuation-in-part of application No. 10/890,041, filed on Jul. 13, 2004.
(60) Provisional application No. 60/487,560, filed on Jul. 14, 2003.

(51) Int. Cl. A61B 17/50

(52) U.S. Cl. 606/131

(57) ABSTRACT

Apparatus for cleaning skin comprising a hand-held body and a head coupled to the body. A vibration generator is disposed within said body and coupled to said head. A pad is removably connected to the head. In certain embodiments, the vibration generator comprises a magnet core rotatably coupled to a motor. A magnet head is fixably coupled to the motor so that the magnet core is disposed within said magnet head. A first magnet is disposed on the magnet core and a second magnet is disposed on the magnet head such that a magnetic field generated by the first magnet interacts with a magnetic field generated by the second magnet as said magnet core rotates.

### CROSS-REFERENCE TO RELATED APPLICATIONS

The present application is a continuation-in-part of and claims priority to U.S. patent application Ser. No. 10/890,041, filed Jul. 13, 2004, and titled “Ultrasonic Skin Cleaner,” which claims priority to U.S. Provisional Application No. 60/487,560, filed Jul. 14, 2003, titled “An Ultrasonic Skin Cleaner,” both of which are hereby incorporated herein by reference for all purposes.

### STATEMENT REGARDING FEDERALLY SPONSORED RESEARCH OR DEVELOPMENT

Not Applicable.

### BACKGROUND

The present invention relates generally to methods and apparatus for cleaning human skin. More particularly, the present invention relates to methods and apparatus that use vibrations to clean human skin.

Ultrasonic vibrations and ultrasound have been used on human skin to perform a variety of tasks, including: softening the thrombus, removing scars after acne is treated, removing abnormal skin color, decomposing abnormal pigment and remove flecks under skin, preventing and removing facial wrinkles and blood stasis, removing “eye-sack” and “black eye-rim,” softening nodules to treat acne, and treating sclerosis and parchment. Ultrasonic vibrations have also been claimed to improve skin tone as well as underlying muscle tone.

In most cases, the application of ultrasonic vibrations to human skin has been done in a spa or clinic setting. These treatments commonly use an ultrasonic transducer that produces high-frequency sound waves up to and including 1 MHz. The ultrasonic transducer transfers vibrations through a metal plate that is applied directly to the skin. The transducer is attached via a cord to a stationary base unit that powers the transducer. A typical treatment may last from 15 to 30 minutes and can cost several hundred to several thousand dollars.

There are also a number of hand-held, home-use devices that employ ultrasound for treating the skin. These hand-held devices include of one or more metal plates that are applied directly to the area of skin that is to be treated. An ultrasonic transducer activates the metal plates and the ultrasonic vibrations pass into the skin and surrounding tissue. These hand-held units are designed to help reduce the visible effects of aging and are sold as a low cost alternative to surgical face-lifts. The units do have great beneficial effects on the overall health of skin, in particular the skin of the face and neck. They can reduce visible wrinkles, smooth scars and increase general muscle tone of under-laying subcutaneous tissue. Unfortunately, some of these units also pose a slight health risk in that the energy that they produce may be damaging to the retina and optic nerve of the human eye, should the user make accidental contact with the eye. This health risk has limited the availability of these products in many countries.

Most of the currently available ultrasonic skin care products are designed to rejuvenate, or improve the overall quality of, the skin but are not specifically designed to act as a daily cleaning appliance. In some treatments ultrasonic energy is used to drive beneficial compounds into the surface of the skin. This is known as “sonophoresis.” However, unwanted impurities found on the surface of the skin may also be driven in by the use of ultrasound. For this reason, it is critical that the surface of the skin to be treated to be free of any foreign material, including, but not limited to, dust, grime, dead skin, excess body oil or other contaminates that may be found on the surface of exposed skin. Therefore, it is often recommended to pre-clean the skin before application of the ultrasonic device as the high frequency sound may in fact drive surface impurities deeper into the skin surface.

Accordingly, it would be desirable to have a skin cleaning system that is intended for daily personal use and operates at a range of frequencies, including ultrasonic frequencies. Thus, there remains a need to develop methods and apparatus for skin cleaning, which overcome some of the foregoing difficulties while providing more advantageous overall results.

### SUMMARY OF THE PREFERRED EMBODIMENTS

The embodiments of the present invention are directed toward Apparatus for cleaning skin comprising a hand-held body and a head coupled to the body. A vibration generator is disposed within said body and coupled to said head. A pad is removably connected to the head. In certain embodiments, the vibration generator comprises a magnet core rotatably coupled to a motor. A magnet head is fixably coupled to the motor so that the magnet core is disposed within said magnet head. A first magnet is disposed on the magnet core and a second magnet is disposed on the magnet head such that a magnetic field generated by the first magnet interacts with a magnetic field generated by the second magnet as said magnet core rotates.

Thus, the present invention comprises a combination of features and advantages that enable it to overcome various problems of prior devices. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is a side elevation view of a skin cleaner constructed in accordance with one embodiment of the invention;

FIG. 2 is front elevation view of the skin cleaner of FIG. 1;

FIG. 3 is a partial cross-sectional view of the skin cleaner of FIG. 1;

FIG. 4 is a top view of a skin cleaner recharging base constructed in accordance with embodiments of the invention;

FIG. 5 is a cross-sectional view of the skin cleaner recharging base of FIG. 4; and

FIG. 6 is a perspective view of an skin cleaner cleaning pad constructed in accordance with embodiments of the invention

FIG. 7 is a skin cleaner constructed in accordance with embodiments of the invention;

FIG. 8 is a cross-sectional elevation view of the skin cleaner of FIG. 7;

FIG. 9 shows the shape of the applicator surface of the skin cleaner of FIG. 7;

FIG. 10 is a cross-sectional elevation view of the drive system of the skin cleaner of FIG. 7;

FIG. 11 is a sectional view of one embodiment of the drive system of FIG. 7; and

FIG. 12 shows one embodiment of a control panel for the skin cleaner of FIG. 7;

### DETAILED DESCRIPTION OF THE PREFERRED EMBODIMENTS

In the description that follows, like parts are marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The present invention is susceptible to embodiments of different forms. There are shown in the drawings, and herein will be described in detail, specific embodiments of the present invention with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results.

In particular, various embodiments described herein thus comprise a combination of features and advantages that overcome some of the deficiencies or shortcomings of prior art skin cleaning systems. The various characteristics mentioned above, as well as other features and characteristics described in more detail below, will be readily apparent to those skilled in the art upon reading the following detailed description of preferred embodiments, and by referring to the accompanying drawings.

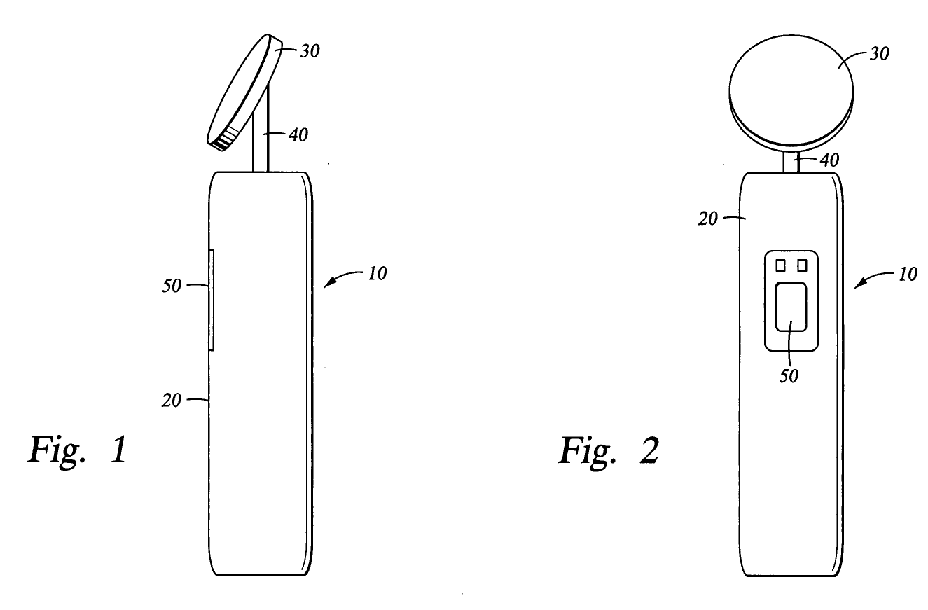

Referring now to FIGS. 1 and 2, skin cleaner 10 comprises main body 20, cleaning head 30, and transfer rod 40. Control panel 50 is mounted on main body 20 and includes a power switch and controls for adjusting the operation of cleaner 10. Main body 20 is sized so as to be hand-held. Cleaning head 30 is at an angle to body 20 in order to allow easy application of the cleaning head to the skin surface. Cleaning head 30 is preferably round in shape, allowing easy contact with contoured skin. Additional shapes for cleaning head 30 may also be used, depending on the desired application.

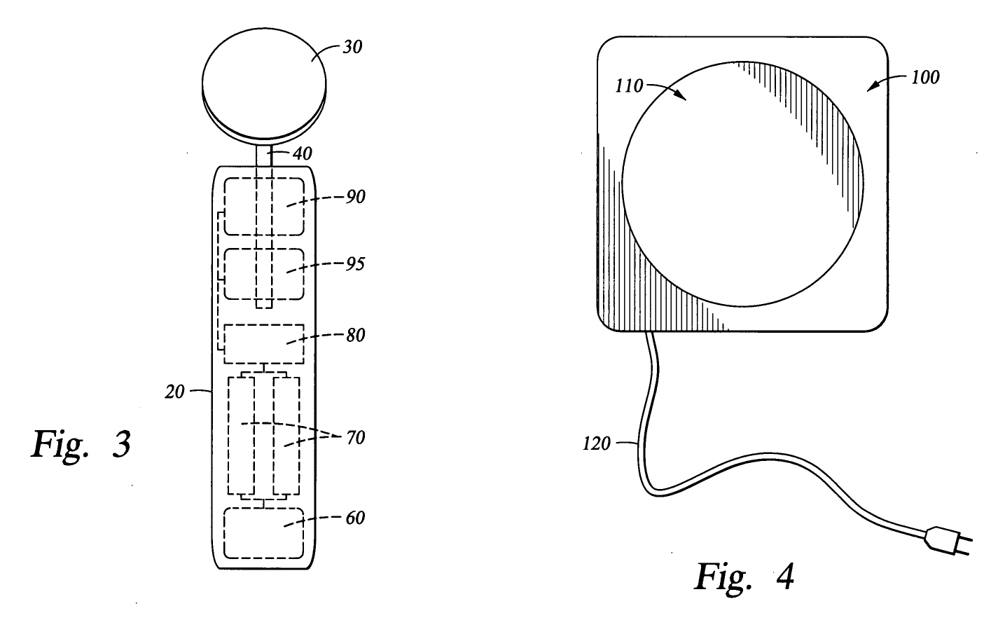

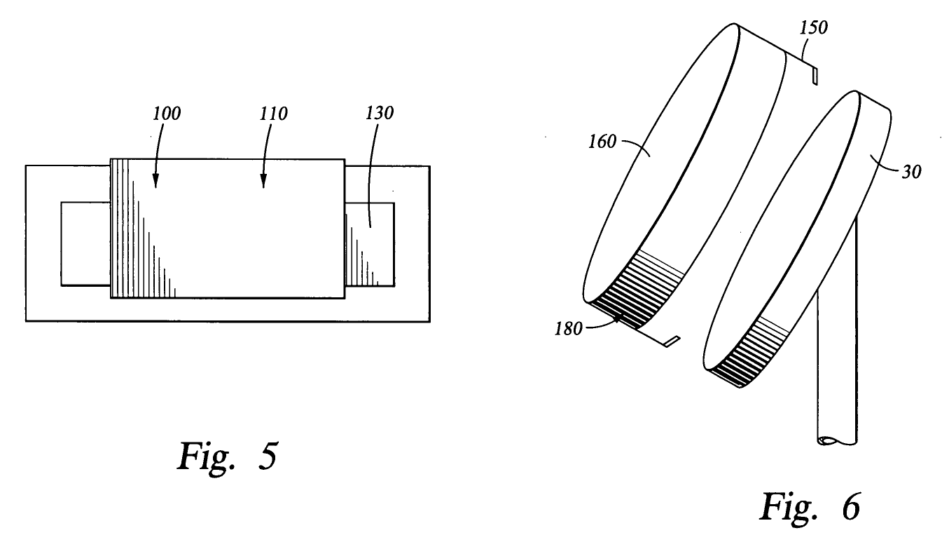

Referring now to FIG. 3, main body 20 houses charger 60, rechargeable batteries 70, electronic control center 80, and vibration generators 90 and 95. Cleaning head 30 is mounted to the protruding end of transfer rod 40. Transfer rod 40 extends into body 20 and is connected to first vibration generator 90 and second vibration generator 95. The frequency and timing of vibration generators 90 and 95 are controlled by electronic control center 80, which receives user input from control panel 50 (see FIGS. 1 and 2). Rechargeable batteries 70 provide power to vibration generators 90 and 95 through electronic control center 80. Charger 60 is coupled to rechargeable batteries 70 and, when connected to a recharging base 100 (see FIGS. 4 and 5), recharges the batteries.

Referring now to FIGS. 4 and 5, recharging base 100 comprises recharging chamber 110, power cord 120, and induction charging coil 130. Recharging chamber 110 provides a recessed area in base 100 that is sized to accept main body 20 (see FIGS. 1-3). Induction charging coil 130 surrounds recharging chamber 110 and provides power to charger 60 when power cord 120 is connected to a power supply (not shown).

Many different skin types require different levels of care in daily cleaning. Providing the optimum daily cleaning regime requires great flexibility in both the type of cleaning agent as well as the physical properties of the cleaning surface. It may be desirous to have a cleaning system that allows the application of other skin care agents, such as moisturizers. These other skin care agents may require a surface pad that is different than that which would be used for cleaning the skin. Further, it is preferred that any cleaning surface be easily cleaned or changed upon each use. Without cleaning or changing, the cleaning surface may harbor a build up of oils and dead skin, thus providing a source of bacterial contamination.

Referring now to FIG. 6, removable cleaning pad 180 comprises attachment ring 150 and cleaning surface 160. Removable cleaning pad 180 attaches to cleaning head 30 via attachment ring 150. Attachment ring 150 may be a compression snap ring easily connected to and disconnected from head 30. Additional attachment methods including an elastic slip cover, groove attachment, and any other form of attachments that would facilitate the easy attachment or removal of cleaning pad 180 to cleaning head 30. Cleaning surface 160 may be made of any natural or artificial fibers or other material as may be suitable for the desired cleaning effect on the skin. The degree of abrasion to the treated skin surface and the depth of the cleaning may be controlled by the nature and composition of the cleaning pad 180.

In operation, a cleaning pad 180 is attached to the cleaning head 30, by attachment ring 150. Cleaning pad 180 may be dampened with water or any desires cleaning agent. Skin cleaner 10 is powered-on by a switch on control panel 50 that activates the electronic control center 80. Control center 80, using power supplied by batteries 70, activates the vibration generators 90 and 95. Vibrations that are generated in the vibration generators 90 and 95 are transferred to the cleaning head 30 and pad 180 via transfer rod 40. Once the unit is powered-on, the surface 160 of cleaning pad 180 is applied to the area of skin that is to be cleaned.

Vibration generators 90 and 95 may be arranged such that their operation causes cleaning head 30 and pad 180 to vibrate in multiple directions. The primary cleaning action of skin cleaner 10 is achieved at the contact between surface 160 of cleaning pad 180 and the surface of the skin. As cleaning pad 180 is applied to the skin, vibrations transferred from cleaning head 30 move the surface 160 of cleaning pad 180 at very high speeds. As cleaning pad 180 is moved back and forth across the area of skin to be cleaned, the vibrations act to lift deep-seated dirt and grime, dead skin cells, and other foreign matter from the skin. In addition, the vibrations may abrade the skin and impart a cleaner fresher look to the treated skin.

All vibrations, including ultrasonic vibrations, cause a displacement in any surface that they react with. This displacement can occur in one or more of three dimensions based on how the vibrations are propagated or generated. In basic terms, the vibrations can cause a surface to move, or be displaced, in a classic x, y, or z orientation in three-dimensional space. For the purpose of cleaning the skin, it is advantageous to have a cleaning surface that is moving or displacing in more than one plane. Specifically, it is advantageous to have the cleaning surface moving in both the x and y axis at the same time. By properly timing the generation of specific vibrations, an orbital pattern of movement can be achieved in the cleaning surface. This orbital motion allows greater movement of the surface of the skin, thus resulting in a greater cleaning action.

Vibration generators 90 and 95 may be used to generate vibrations in two planes and thus cause transfer rod 40 to move the cleaning head 30 in multiple dimensions during use. Adjusting the timing of the vibrations in the multiple planes during operation causes a displacement of cleaning head 30 and cleaning pad 180, which is in contact with the skin. Vibration generators 90 and 95 may be transducers, unbalanced electric motors, electromagnets, or any other device capable of generating high-frequency vibrations, including ultrasonic vibrations at over 1000 cycles per second. In certain embodiments, a single vibration generator may be used to produce unidirectional motion.

Control center 80 may provide for a timed or non-timed treatment period as may be desired by the user. Thus, when electronic control center 80 switches cleaner 10 off, the cleaning pad 180 may be removed for cleaning or replacement. After use, cleaner 10 may be placed into charging base 100 such that rechargeable batteries 70 can be recharged by charger 60 and induction charger coil 130. As an alternative to rechargeable batteries 70, certain embodiments of cleaner 10 may be equipped with replaceable batteries or be connectable to an external power supply.

As described herein, skin cleaner 10 provides a multi-directional skin cleaning appliance comprising a hand held body 20 having a rechargeable battery 70 and a removable and/or replaceable cleaning pad 180. Body 20 and recharging base 100 may also preferably be freestanding and waterproof so as to enable easy and safe storage in the home environment. Thus, skin cleaner 10 allows a user to deep clean the surface of the skin in the privacy of their home on a daily basis. Advantages to this type of deep cleaning would assist in the control of acne, the removal of blemishes, reduction of scar tissue and improvement to the general overall health of the skin that is treated.

In other embodiments, skin cleaner 10 may have a removable and interchangeable cleaning head. A removable cleaning head could be removed from the transfer rod and replaced with an alternate cleaning head having another shape. The alternate cleaning head would also carry the vibrations from the transfer rod. Interchangeable cleaning heads could also support multiple uses of a single drive unit. For example, one alternate head may be a shaving head including a blade holder. Another alternate head may comprise a shaped, abrasive pad for use in nail care for shaping and/or buffing the nail. Alternate heads may also include heating elements, high-intensity magnets, or other features to improve cleaning performance.

In another embodiment, a skin cleaner may have a transfer rod that is turned at a 90 from the drive units and have a cleaning head that floats on a gasket. The cleaning head could support removable cleaning pads. This configuration would produce a device that is designed for cleaning large areas of skin. The device would preferably water-proof and could be designed to look similar to a bar of soap.

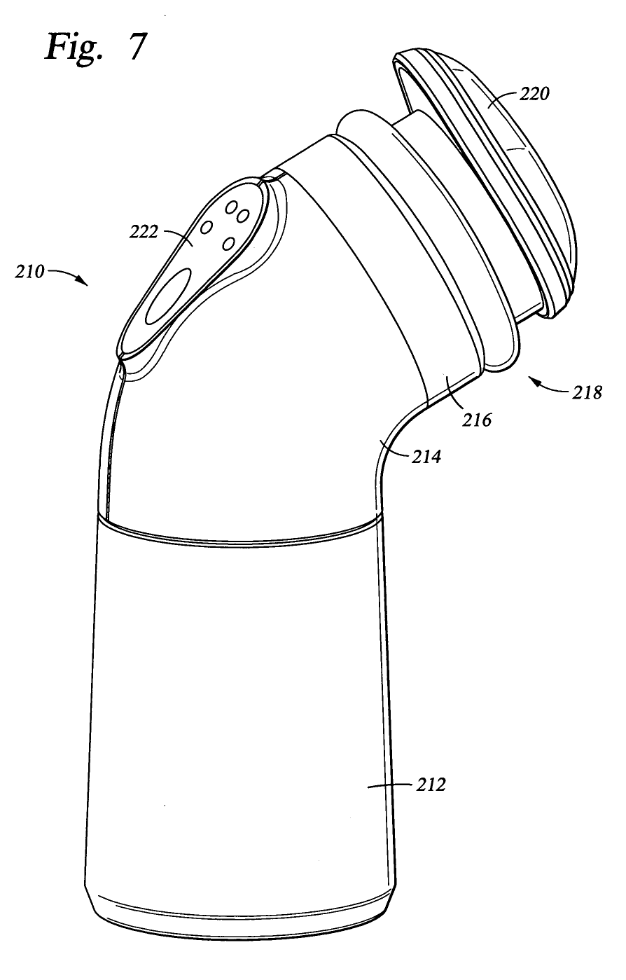

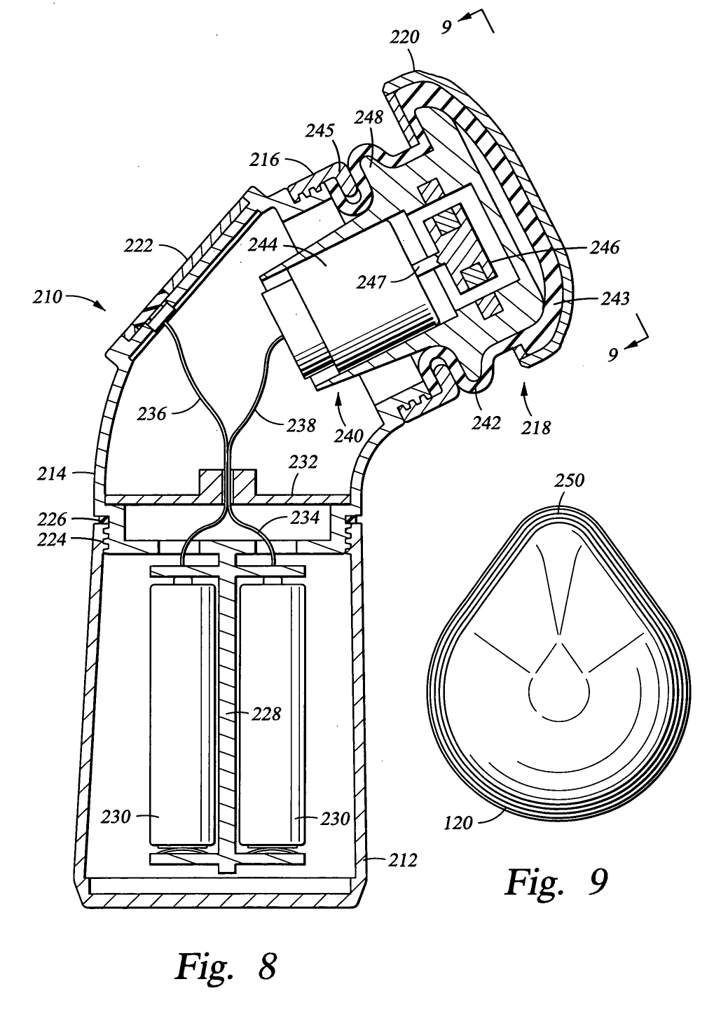

Referring now to FIGS. 7 and 8, skin cleaner 210 comprises base 212, body 214, head nut 216, vibrating head assembly 218, detachable cleaning pad 220, and control panel 222. Base 212 is coupled to one end of body 214. Vibrating head assembly 218 is flexibly coupled to body 214 by head nut 216. Cleaning pad 220 is detachably connected to vibrating head assembly 218. Control panel 222 is mounted to body 214 and provides user control of skin cleaner 210. When skin cleaner 210 is activated, head assembly 218 and cleaning pad 220 are vibrated at a selectable frequency, up to and exceeding ultrasonic frequencies, e.g. at least 1000 vibration cycles per second, by a vibration generator 240 (see FIG. 8) disposed within the body.

Base 212 is coupled to body 214 via threads 224. Seal 226 forms a water-tight seal between base 212 and body 214. Base 212 and body 214 are formed so as to allow ergonomic application of cleaning pad 220 to a user's skin. Body 214 and base 212 may be sized so that skin cleaner 210 is free-standing and hand-held. Body 214 further comprises battery support 228 that extends from body 214 and supports batteries 230 in a volume surrounded by base 212.

Circuit board 232 is also supported within body 214 and is coupled to batteries 230 via wires 234. Circuit board 232 also couples to control panel 222 via wires 236 and to vibration generator 240 via wires 238. Circuit board 232 comprises the electronic circuitry used to control the operation of vibration generator 240 and manage the power drawn from batteries 230. In certain embodiments, circuit board 232 may draw power from each of the plurality of batteries 230 at an equal rate such that a single battery is not drained faster than any of the other batteries. Batteries 230 are illustrated as conventional replaceable batteries but in other embodiments, the batteries may be replaced by custom made rechargeable batteries or some other electrical power source.

As shown in FIG. 8, vibrating head assembly 218 comprises vibration generator 240 and flexible membrane 242. Vibration generator 240 comprises motor 244, magnet core 246, and magnet head 248. Flexible membrane 242 substantially surrounds vibration generator 240 and further comprises applicator surface 243 and flange 245. Flange 245 engages head nut 216 so as to provide a flexible attachment of vibrating head assembly 218 to body 214. This flexible attachment serves to partially isolate the vibrations within head assembly 218 from the body. Cleaning pad 220 substantially covers applicator surface 243 so that the cleaning pad contacts the skin being treated while flexible membrane 242 acts as a compliant substrate that supports the pad and transfers vibrations into the pad.

The shape of applicator surface 243 and cleaning pad 220 can be seen in FIG. 9. In the embodiment shown, magnet head 248, flexible membrane 242, and cleaning pad 220 are constructed so that applicator surface 243 and cleaning pad 220 have at least one reduced radius portion 250. Reduced radius portion 250 allows cleaning pad 220 to access small areas of the skin that a completely round cleaning pad could not reach as easily. Reduced radius portion 250 also acts to concentrate and reflect vibrations across vibrating head 218. These reflected vibrations enhance the cleaning action of cleaning pad 220. In the embodiment shown in FIG. 8, applicator surface 243 and cleaning pad 220 have a tear-drop shape with a single reduced radius portion 250. In other embodiments, two or more reduced radius portions, which may have different sizes, may also be used to achieve the desired vibration behavior and cleaning results.

Cleaning pad 220 is preferably constructed from a microfiber material sized so as to closely and securely fit on flexible membrane 242. Cleaning pad 220 is preferably removable from flexible membrane 242 so as to allow removal of the pad for cleaning or replacement. Flexible membrane 242 may be constructed from a pliable material that transfers vibrations into cleaning pad 220 while providing a soft but stable underlayment for the pad and a secure attachment to body 214. Flexible membrane 242 may also be non-permeable so as to ease cleaning and provide a water-tight seal with body 214. In certain embodiments, flexible membrane 242 is constructed from a silicone based material.

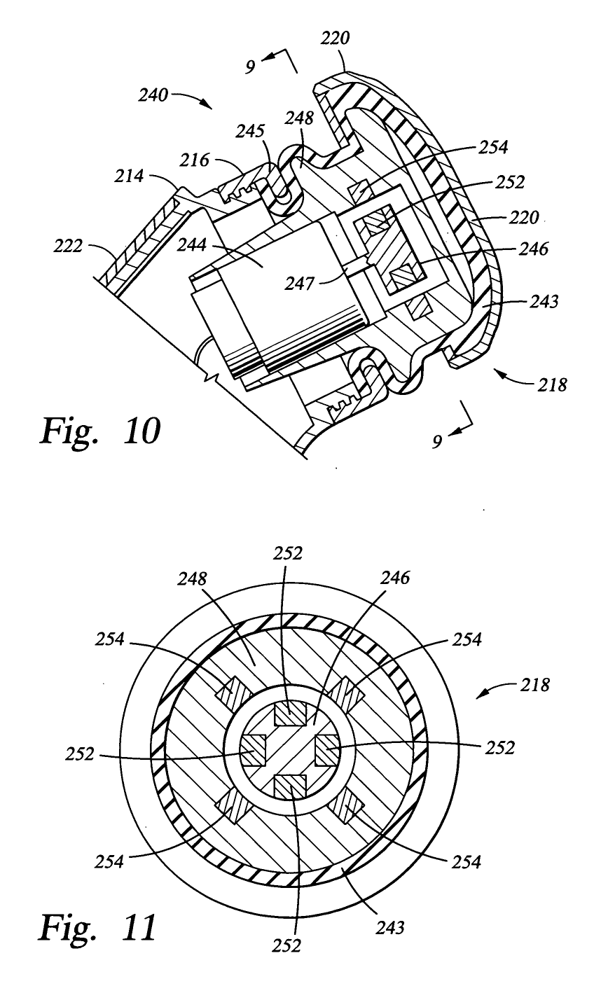

As is further shown in FIG. 10, vibration generator 240 comprises motor 244, magnet core 246, and magnet head 248. Magnets 252, 254 are mounted to magnet core 246 and magnet head 248, respectively. Magnet core 246 is mounted to motor shaft 247 such that the operation of motor 244 results in the rotation of the magnet core relative to magnet head 248. Motor 244 is mounted so as to be stationary relative to magnet head 248. Magnets 252 and 254 are preferably high-strength permanent magnets, such as 2500 gauss samarium cobalt magnets. Motor 244 may be a variable-speed electric motor that operates at up to 1500 revolutions per minute with sufficient torque to rotate core 246 within head 248.

In operation, motor 244 rotates magnet core 246 relative to magnet head 248. As magnet core 246 rotates relative to magnet head 248, the magnetic fields of magnets 252 into and out of the magnetic fields of magnets 254. The magnets are arranged so that the positive and negative poles of the magnets interact in a non-uniform manner. This interaction generates varying attractive and repulsive forces between the magnets 252, 254. These varying forces cause vibrations in vibration generator 240.

In general, as the number of magnets increases, the number of vibrations per revolution of the motor also increases. Thus, the frequency of the vibrations can be increased by increasing the rotational speed of magnet core 246 via motor 244. The frequency and magnitude of the vibrations can also be controlled by varying the strength, placement, and selection of magnets 252 and 254. In certain embodiments, vibration generator 240 generates vibrations within a range of frequencies up to a maximum frequency of more than 1000 vibrations per second.

In the embodiment shown in FIG. 11, magnet core 246 comprises four magnets 252 evenly spaced around the core. Magnets 252 are arranged such that the positive poles of two opposed magnets face magnet head 248 while the other two magnet's negative poles face the magnet head. Magnets 254 in magnet head 248 are arranged in a similar alternating-pole manner but are not spaced evenly about the head. In certain embodiments, magnets 254 are spaced approximately 68° apart.

Other arrangements and quantities of magnets are possible and may be used to achieve a desired vibration frequency and magnitude. For skin cleaning applications, it may be preferable to minimize the magnitude of the vibrations while maximizing the frequency. Other applications may benefit from other combinations of frequency and magnitude.

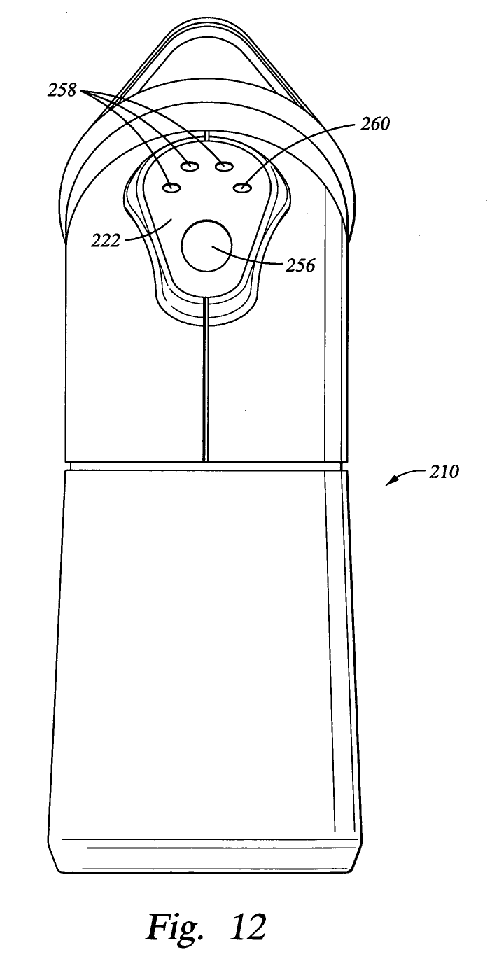

Referring now to FIG. 12, one embodiment of control panel 222 is shown comprising activation switch 256, mode indicating light emitting diodes (LED's) 258, and battery charge indicating LED 260. Circuit board 232 comprises a microcontroller programmed to operate cleaner 210 in one of three modes according to a selection made by a user through activation switch 256. In each of the three modes, skin cleaner 210 may operate at a different vibration frequency so as to benefit the performance of a given cleaning task. For example, skin cleaner 210 may operate in a deep cleaning mode at ultrasonic frequencies, a daily cleaning mode at less than ultrasonic frequencies, and a moisturizer application mode at a lower frequency.

When a user first presses activation switch 256, motor 244 is activated at full power so as to overcome forces from magnets 252 and 254 that seek to keep the motor stationary. Power to motor 244 is then decreased and cleaner 210 will operate in a first mode for a predetermined period of time. Pressing activation switch 256 a second time will shift cleaner 210 into a second mode for a predetermined amount of time and pressing the activation switch a third time will shift the cleaner into a third mode for a predetermined amount of time. The mode in which cleaner 210 is operating will be indicated by the activation of one of LED's 258. After operating for the predetermined period of time, cleaner 10 will deactivate until the user presses activation switch 256.

While preferred embodiments of this invention have been shown and described, modifications thereof can be made by one skilled in the art without departing from the scope or teaching of this invention. The embodiments described herein are exemplary only and are not limiting. Many variations and modifications of the system and apparatus are possible and are within the scope of the invention. For example, the relative dimensions of various parts, the materials from which the various parts are made, and other parameters can be varied, so long as the systems retain the advantages discussed herein. Accordingly, the scope of protection is not limited to the embodiments described herein, but is only limited by the claims that follow, the scope of which shall include all equivalents of the subject matter of the claims.

What is claimed is:

1. An apparatus for treating skin comprising:
a hand-held body;
a head coupled to said body;
a vibration generator disposed within said body and coupled to said head; and
a pad removably connected to said head.

2. The apparatus of claim 1, wherein said head further comprises a flexible membrane having an applicator surface, wherein said pad covers the applicator surface of said flexible membrane.

3. The apparatus of claim 2 wherein said head is coupled to said body by a head nut that engages said body and said flexible membrane.

4. The apparatus of claim 1 wherein said head is flexibly coupled to said body.

5. The apparatus of claim 1 wherein said pad comprises at least one region of reduced radius.

6. The apparatus of claim 1 further comprising a power source supported by said body and electrically coupled to said vibration generator.

7. The apparatus of claim 6 wherein said power source is disposed within a base that is coupled to said body.

8. The apparatus of claim 1 wherein said vibration generator further comprises:
a motor;
a magnet core rotatably coupled to said motor and having a first magnet disposed thereon; and
a magnet head fixably coupled to said motor and having a second magnet disposed thereon, wherein said magnet core is disposed within said magnet head.

9. The apparatus of claim 8 wherein a magnetic field generated by the first magnet interacts with a magnetic field generated by the second magnet as said magnet core rotates.

10. A skin cleaning device comprising:
a vibration generator disposed within a body;
a vibrating head coupled to said vibration generator and flexibly coupled to the body; and
a pad removably disposed on said vibrating head.

11. The skin cleaning device of claim 10, wherein said vibrating head is covered by a flexible membrane having an applicator surface, wherein said pad is removably disposed on the applicator surface of said flexible membrane.

12. The apparatus of claim 11 wherein said vibrating head is flexibly coupled to said body by a head nut that engages said body and said flexible membrane.

13. The skin cleaning device of claim 10, wherein said vibrating head comprises an applicator surface having at least one region of reduced radius.

14. The skin cleaning device of claim 10 wherein said vibration generator further comprises:
a motor;
a magnet core rotatably coupled to said motor and having a first magnet disposed thereon; and
a magnet head fixably coupled to said motor and having a second magnet disposed thereon, wherein said magnet core is disposed within said magnet head.

15. The skin cleaning device of claim 14 wherein a magnetic field generated by the first magnet interacts with a magnetic field generated by the second magnet as said magnet core rotates.

16. An apparatus comprising:
means for attaching a pad to a cleaning device;
means for generating vibrations in the pad; and
means for removing the pad from the cleaning device.

17. The apparatus of claim 16 wherein the pad further comprises means for concentrating and reflecting vibrations in the pad.

18. The apparatus of claim 16 further comprising means for supplying power to the means for generating vibrations.

19. The apparatus of claim 16 wherein the cleaning device is a hand held device.

20. The apparatus of claim 16 further comprising means for flexibly connecting the pad to the cleaning device.
